(19) United States
(12) Patent Application Publication
(10) Pub. No.: US 2021/0408398 A1
(43) Pub. Date: Dec. 30, 2021

(54) HOLE TRANSPORT MATERIAL, PREPARATION METHOD THEREOF, AND ORGANIC LIGHT-EMITTING DEVICE

(71) Applicant: WUHAN CHINA STAR OPTOELECTRONICS SEMICONDUCTOR DISPLAY TECHNOLOGY CO., LTD, Wuhan (CN)

(72) Inventors: Jiajia LUO, Wuhan (CN); Munjae LEE, Wuhan (CN); Xianjie Li, Wuhan (CN); Xu WANG, Wuhan (CN); Yan LI, Wuhan (CN); Yuanyuan ZHANG, Wuhan (CN)

(73) Assignee: WUHAN CHINA STAR OPTOELECTRONICS SEMICONDUCTOR DISPLAY TECHNOLOGY CO., LTD, Wuhan (CN)

(21) Appl. No.: 16/755,574

(22) PCT Filed: Dec. 3, 2019
(86) PCT No.: PCT/CN2019/122753

§ 371 Date: Apr. 11, 2020

(30) Foreign Application Priority Data
Nov. 25, 2019 (CN) 201911162480.0

(51) Int. Cl. H01L 51/00; C07D 493/10; C09K 11/06

(52) U.S. Cl. CPC H01L 51/0073; C07D 493/10; H01L 51/5056; H01L 51/0072; C09K 11/06

(57) ABSTRACT

The present invention discloses a hole transport material, a preparation method thereof, and an organic light-emitting device. The hole transport material includes a compound having a chemical structure represented by Formula 1:

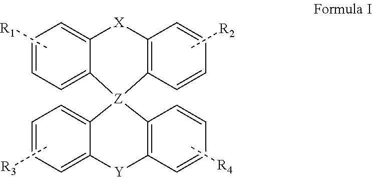

wherein Z includes a carbon atom or a silicon atom, each of X and Y independently includes: an oxygen atom, a sulfur atom,

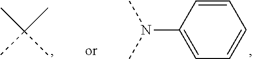

any one or two of R1, R2, R3, and R4 are electron-donors, and remaining of R1, R2, R3, and R4 other that the electron-donors are hydrogen atoms.

### BACKGROUND OF INVENTION

### Field of Invention

The present invention relates to the field of display technology, and in particular, to a hole transport material, a preparation method thereof, and an organic light-emitting device.

### Description of Prior Art

It is known that organic light-emitting diodes (OLEDs) have attracted attention from many researchers, due to their huge application prospects and advantages, such as self-illumination without the need for a backlight, high light-emitting efficiency, wide viewing angles, fast response speed, a large temperature adaptation range, relatively simple production and processing techniques, low driving voltage, low energy consumption, lightness, thinness, flexibility, and so on.

In an OLED, according to functions, it can be divided into hole injection material, hole transport material, light-emitting material, electron transport material, electron injection material, metal cathode. The currently used top-emitting OLED devices include the hole transport material as the thickest layer, and there has always been a contradiction between its energy level and hole mobility. Therefore, there is an urgent need to develop a hole transport material having a matched energy level and high mobility.

Embodiments of the present invention provide a hole transport material, a preparation method thereof, and an organic light-emitting device, so as to solve the technical problems of low hole mobility and mismatched energy levels of the existing hole transport materials.

### SUMMARY OF INVENTION

The present invention provides a hole transport material, a preparation method thereof, and an organic light-emitting device, which can improve the mobility of the hole transport material, so as to solve the technical problems of low hole mobility and mismatched energy levels of the existing hole transport materials, thereby impacting display.

To solve the above problems, the technical solution provided by the present invention is as follows:

The present invention provides a hole transport material, which includes a compound having a chemical structure represented by Formula I:

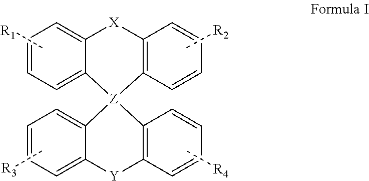

wherein Z includes a carbon atom or a silicon atom, each of X and Y independently includes: an oxygen atom, a sulfur atom,

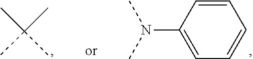

any one or two of R1, R2, R3, and R4 are electron-donors, and remaining of R1, R2, R3, and R4 other that the electron-donors are hydrogen atoms.

2. The hole transport material according to claim 1, wherein each of R1, R2, R3, and R4 independently is or substitutes any hydrogen atom on its corresponding benzene ring.

3. The hole transport material according to claim 1, wherein each of the any one or two of R1, R2, R3, and R4 independently includes a carbazolyl or a derivative thereof, diphenylamino or a derivative thereof, phenoxazinyl or a derivative thereof, or acridinyl or a derivative thereof.

4. The hole transport material according to claim 3, wherein each of the any one or two of R1, R2, R3, and R4 independently includes any one of the following groups:

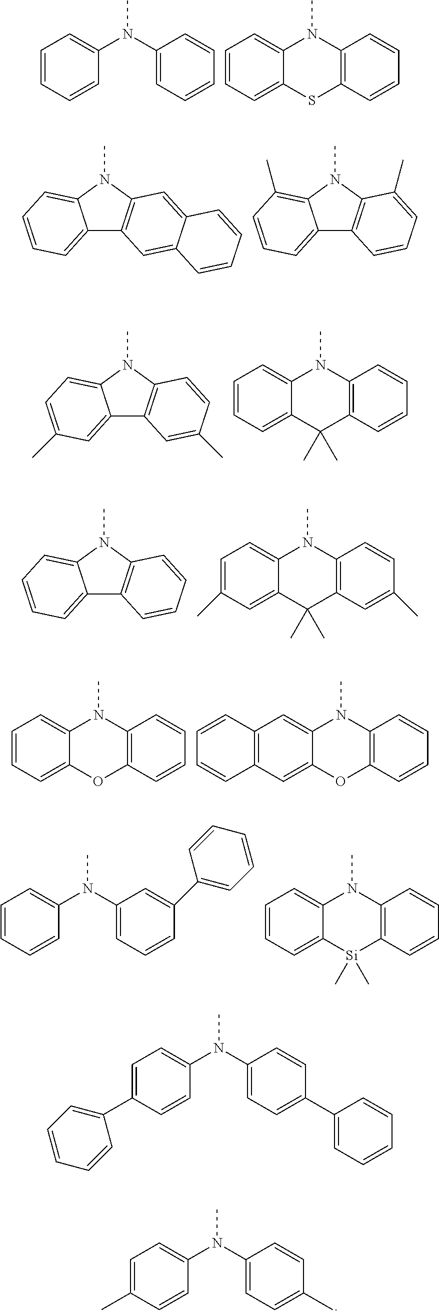

According to the above object of the present invention, a method of preparing a hole transport material is provided, which includes the following steps:

under a protective gas atmosphere, adding a catalyst to any one or two hydrides of R1, R2, R3, and R4 and halospiroxanthene, which are dissolved in an organic solvent to perform a coupling reaction, to obtain a compound having a chemical structure represented by Formula I:

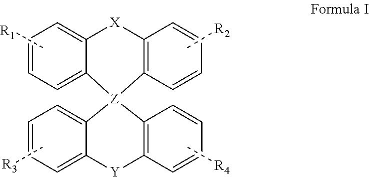

wherein Z includes a carbon atom or a silicon atom, each of X and Y independently includes: an oxygen atom, a sulfur atom,

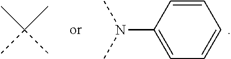

any one or two of R1, R2, R3, and R4 corresponding to the hydrides are electron-donors, and remaining of R1, R2, R3, and R4 other that the electron-donors are hydrogen atoms.

In one embodiment of the present invention, each of the any one or two of R1, R2, R3, and R4 corresponding to the hydrides independently includes a carbazolyl or a derivative thereof, diphenylamino or a derivative thereof, phenoxazinyl or a derivative thereof, or acridinyl or a derivative thereof.

In one embodiment of the present invention, each of the any one or two of R1, R2, R3, and R4 corresponding to the hydrides independently includes any one of the following groups:

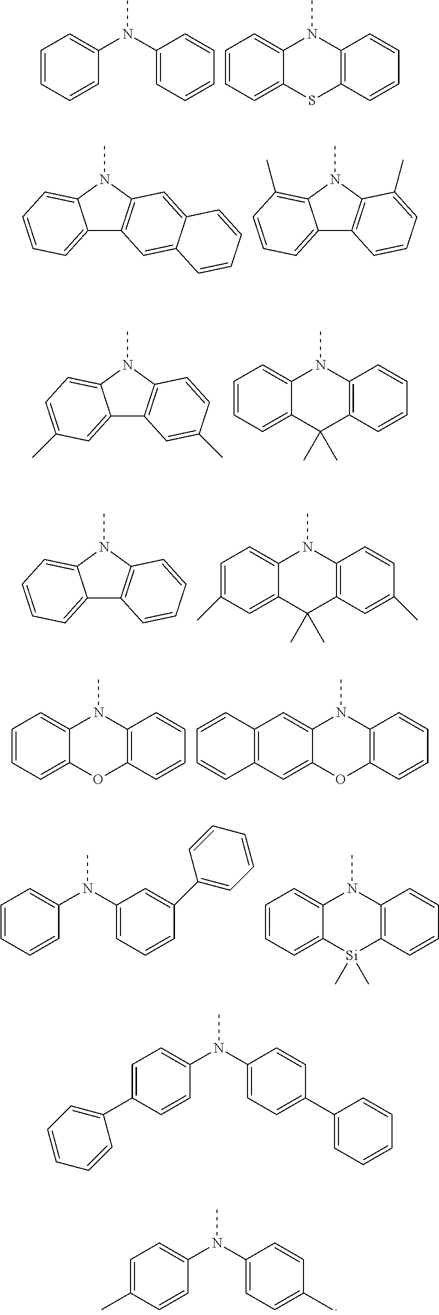

In one embodiment of the present invention, a molar ratio of the halospiroxanthene to the hydrides ranges from 1:1 to 1:3, and the method further includes the following steps:

adding a first catalyst, a second catalyst, and a basic substance during the coupling reaction; and

after the coupling reaction is completed, obtaining the compound by cooling, extraction, and column chromatography.

In one embodiment of the present invention, wherein the first catalyst includes a divalent palladium catalyst, the second catalyst includes tri-tert-butylphosphine tetrafluoroborate, the basic substance includes sodium tert-butoxide or potassium tert-butoxide, and the organic solvent includes dehydrated and deoxygenated toluene.

According to the above object of the present invention, an organic light-emitting device is provided, which includes a first electrode, a second electrode, and a functional layer disposed between the first electrode and the second electrode, and the functional layer includes a hole transport material,

wherein the functional layer includes:

a hole injection layer disposed on the first electrode;

a hole transport layer including the hole transport material and disposed on the hole injection layer;

a light-emitting layer disposed on the hole transport layer;

an electron transport layer disposed on the light-emitting layer; and

an electron injection layer disposed on the electron transport layer;

wherein the hole transport material includes a compound having a chemical structure represented by Formula I:

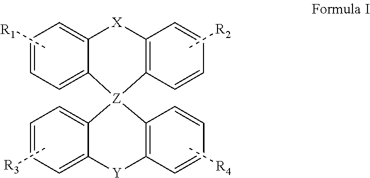

wherein Z includes a carbon atom or a silicon atom, each of X and Y independently includes: an oxygen atom, a sulfur atom,

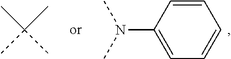

any one or two of R1, R2, R3, and R4 are electron-donors, and remaining of R1, R2, R3, and R4 other that the electron-donors are hydrogen atoms.

In one embodiment of the present invention, each of R1, R2, R3, and R4 independently is or substitutes any hydrogen atom on its corresponding benzene ring.

In one embodiment of the present invention, each of the any one or two of R1, R2, R3, and R4 independently includes a carbazolyl or a derivative thereof, diphenylamino or a derivative thereof, phenoxazinyl or a derivative thereof, or acridinyl or a derivative thereof.

In one embodiment of the present invention, each of the any one or two of R1, R2, R3, and R4 independently includes any one of the following groups:

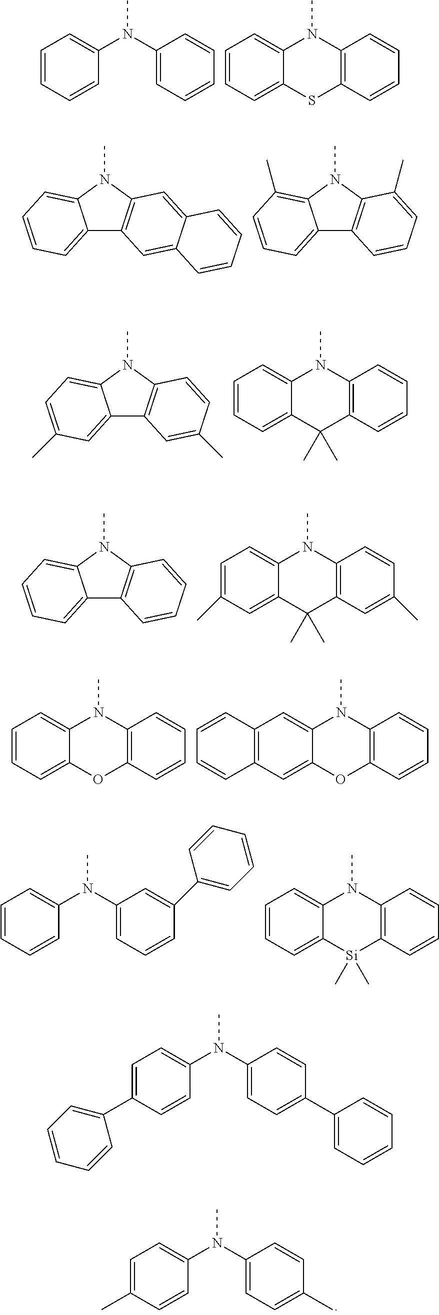

The present invention introduces spiroxanthracene as a core, which is combined with other electron donors, to provide a hole transport material, so that the hole transport material has a higher hole mobility and a suitable energy level, such that the light-emitting efficiency of the organic light-emitting device is improved, and the display effect is elevated.

### BRIEF DESCRIPTION OF DRAWINGS

In order to more clearly illustrate the embodiments or the technical solutions of the existing art, the drawings illustrating the embodiments or the existing art will be briefly described below. Obviously, the drawings in the following description merely illustrate some embodiments of the present invention. Other drawings may also be obtained by those skilled in the art according to these FIGURES without paying creative work.

FIG. 1 is a schematic structural diagram of an organic light-emitting device according to an embodiment of the present invention.

### DETAILED DESCRIPTION OF PREFERRED EMBODIMENTS

Please refer to the FIGURES in the drawings, in which, like numbers refer to like elements throughout the description of the FIGURES. Hereinafter, the present invention will be described in further detail with reference to examples. It is to be understood, however, that these examples are for illustrative purposes only and are not intended to limit the scope of the present invention.

In the description of the present invention, it is to be understood that the terms “center”, “longitudinal”, “transverse”, “length”, “width”, “thickness”, “upper”, “lower”, “front”, “post”, “left”, “right”, “vertical”, “horizontal”, “top”, “bottom”, “inside”, “outside”, “clockwise counterclockwise” etc. demonstrating the orientation or positional relationship of the indications is based on the orientation shown in the drawings Or, the positional relationship is merely for the convenience of the description of the present invention and the simplification of the description, and is not intended to imply that the device or the component of the present invention has a specific orientation and is constructed and operated in a specific orientation, thus being not to be construed as limiting the present invention. Moreover, the terms “first” and “second” are used for descriptive purposes only and are not to be construed as indicating or implying a relative importance or not to implicitly indicate a number of technical features indicated. Thus, features defined by “first” or “second” may include one or more of the described features either explicitly or implicitly. In the description of the present invention, the meaning of “a plurality” is two or more unless specifically defined otherwise.

In the description of the present invention, it should be noted that the terms “installation”, “connection”, and “bonding” are to be understood broadly unless otherwise explicitly defined and limited. For example, it may be fixed connection, detachable connection, or integrally connection; being mechanical or electrical connection; also, being directly connection, indirectly connection through an intermediate medium, or internal communication of two components. The specific meaning of the above terms in the present invention can be understood in a specific case by those skilled in the art.

In the present invention, unless otherwise expressly stated and limited, the formation of a first feature over or under a second feature in the description that follows may include embodiments in which the first and second features are formed in direct contact, and may also include embodiments in which additional features may be formed between the first and second features, such that the first and second features may not be in direct contact. Moreover, the first feature “above”, “over” and “on” the second feature includes the first feature directly above and above the second feature, or merely indicating that the first feature is at a level higher than the second feature. The first feature “below”, “under” and “beneath” the second feature includes the first feature directly below and obliquely below the second feature, or merely the first feature has a level lower than the second feature.

The following disclosure provides many different embodiments or examples for implementing different structures of the present invention. In order to simplify the disclosure of the present invention, the components and arrangements of the specific examples are described below. Of course, they are merely examples and are not intended to limit the present invention. In addition, the present invention may repeat reference numerals and/or reference letters in the various embodiments, which are for the purpose of simplicity and clarity, and do not indicate the relationship between the various embodiments and/or arrangements discussed. Moreover, the present invention provides examples of various specific processes and materials, but one of ordinary skill in the art will recognize the use of other processes and/or the use of other materials.

The present invention provides a hole transport material, a preparation method thereof, and an organic light-emitting device, which can improve the mobility of the hole transport material, so as to solve the technical problems of low hole mobility and mismatched energy levels of the existing hole transport materials, thereby impacting display.

resent invention provides a hole transport material, which includes a compound having a chemical structure represented by Formula I:

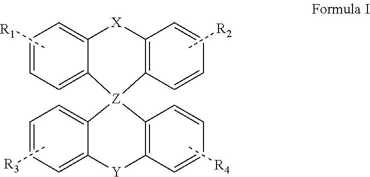

wherein Z includes a carbon atom or a silicon atom, each of X and Y independently includes: an oxygen atom, a sulfur atom,

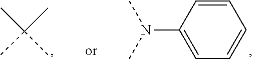

any one or two of R1, R2, R3, and R4 are electron-donors, and remaining of R1, R2, R3, and R4 other that the electron-donors are hydrogen atoms.

In specific implementation, an embodiment of the present invention introduces spiroxanthracene as a core, which is combined with other electron donors, to provide a hole transport material, so that the hole transport material has a higher hole mobility and a suitable energy level, such that the light-emitting efficiency of the organic light-emitting device is improved, and the display effect is elevated.

Further, each of the any one or two of R1, R2, R3, and R4 independently includes a carbazolyl or a derivative thereof, diphenylamino or a derivative thereof, phenoxazinyl or a derivative thereof, or acridinyl or a derivative thereof.

Specifically, each of the any one or two of R1, R2, R3, and R4 independently includes any one of the following groups:

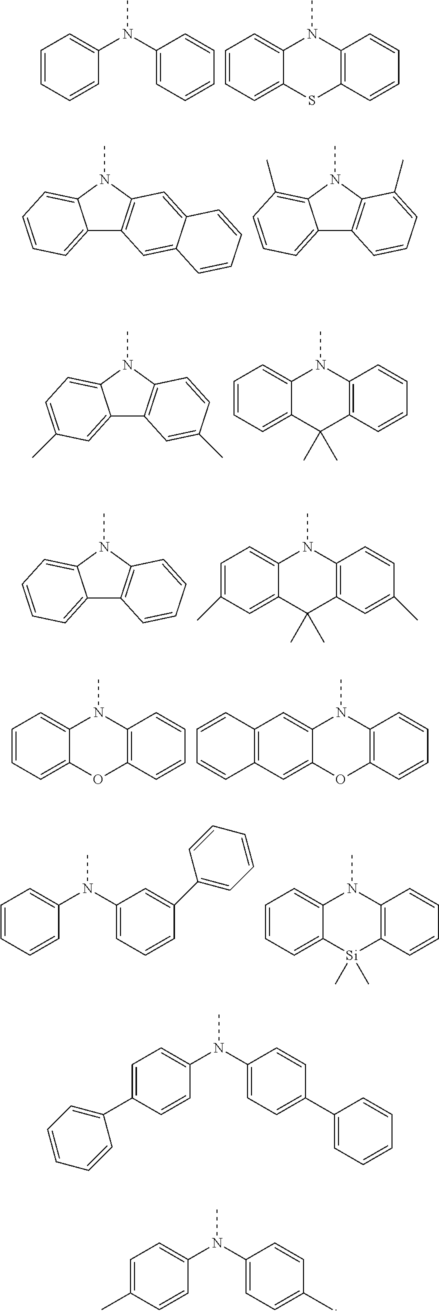

In addition, each of R1, R2, R3, and R4 independently is or substitutes any hydrogen atom on its corresponding benzene ring, that is, each of the electron donors in R1, R2, R3, and R4 can substitute any hydrogen atom on its corresponding benzene ring, and each of the hydrogen atoms in R1, R2, R3, and R4 can also be any hydrogen atom on its corresponding benzene ring.

In addition, a central core compound of the compound, excluding the electron donors, includes any one of the following 20 compounds:

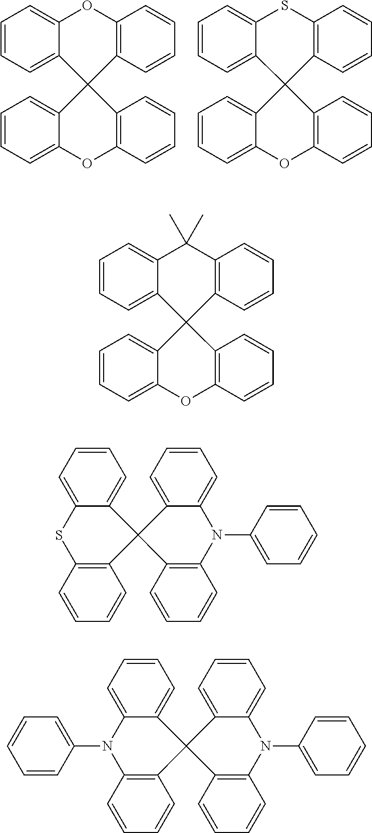

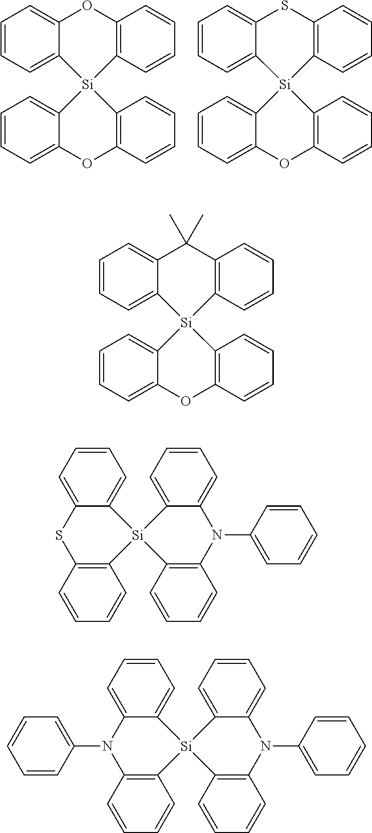

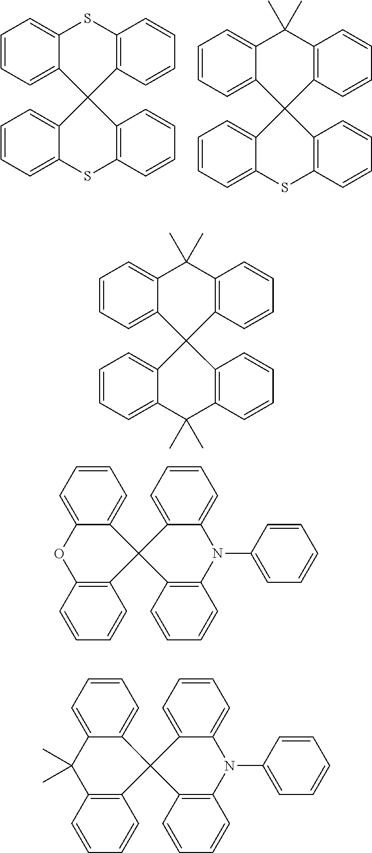

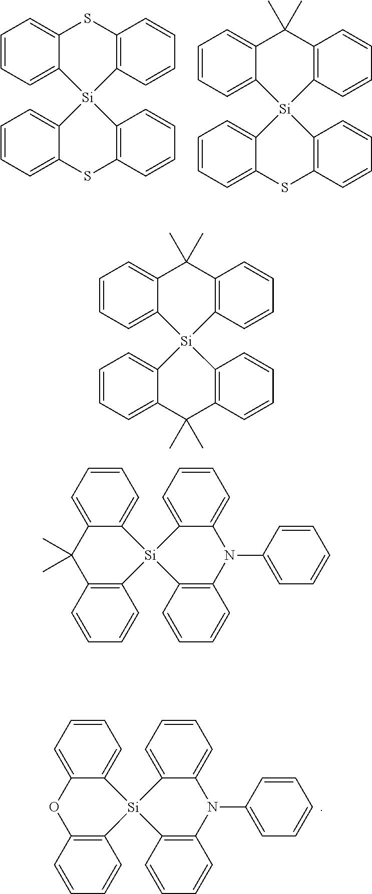

In the following, substitution of the electron donors in the compound provided in embodiments of the present invention will be exemplarily illustrated.

Taking

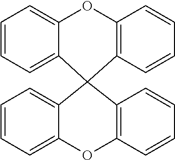 [SMILES: c1ccc2c(c1)Oc1ccccc1C21c2ccccc2Oc2ccccc21]

as the central core compound, with carbazolyl as the electron donors an example for explanation.

When R1 is the electron donor, R2, R3, and R4 are all hydrogen atoms,

the compound includes any one of:

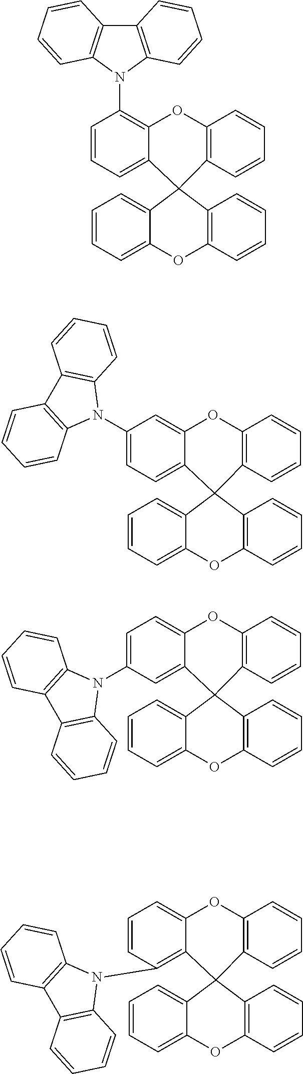 [SMILES: c1ccc2c(c1)Oc1ccccc1C21c2ccccc2Oc2c(-n3c4ccccc4c4ccccc43)cccc21 ; c1ccc2c(c1)Oc1ccccc1C21c2ccccc2Oc2cc(-n3c4ccccc4c4ccccc43)ccc21 ; c1ccc2c(c1)Oc1ccccc1C21c2ccccc2Oc2ccc(-n3c4ccccc4c4ccccc43)cc21 ; c1ccc2c(c1)Oc1ccccc1C21c2ccccc2Oc2cccc(-n3c4ccccc4c4ccccc43)c21]

When R1 and R2 are the electron donors, and R3 and R4 are hydrogen atoms,

the compound includes any one of:

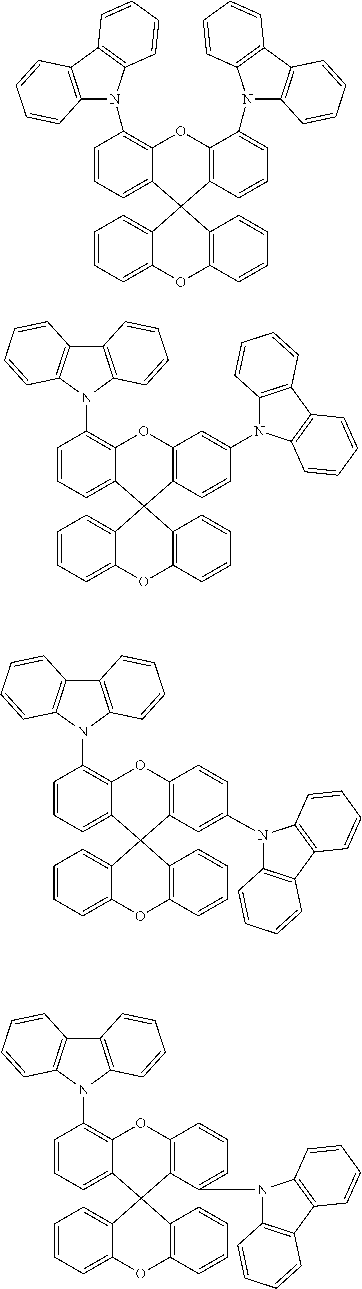

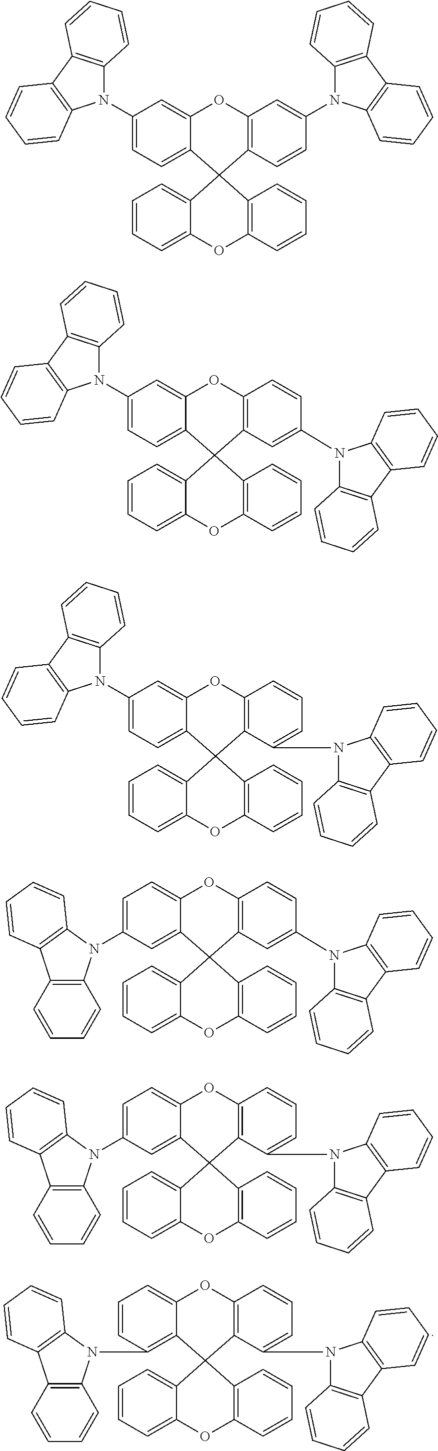

When R1, R4 are the electron donors, and R2 and R3 are hydrogen atoms,

the compound includes any one of:

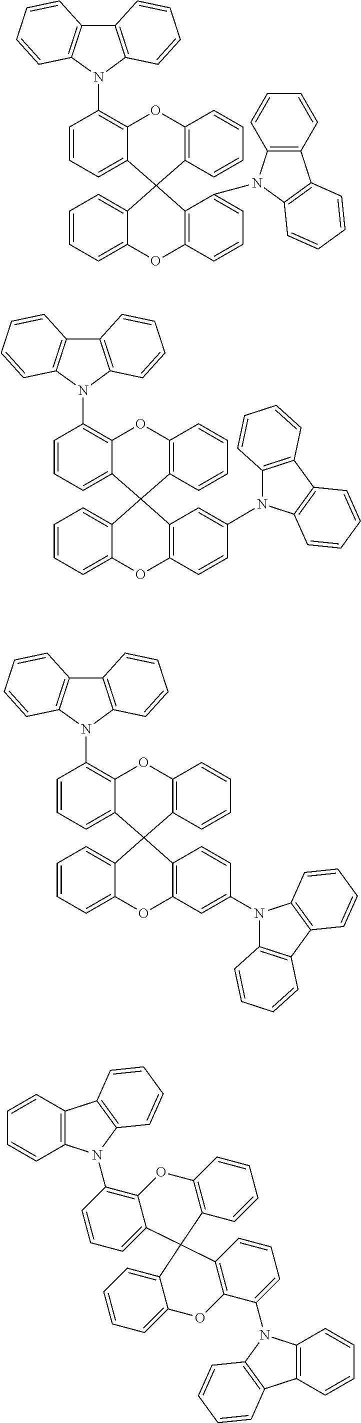 [SMILES: c1ccc2c(c1)Oc1cccc(-n3c4ccccc4c4ccccc43)c1C21c2ccccc2Oc2c(-n3c4ccccc4c4ccccc43)cccc21 ; c1ccc2c(c1)Oc1ccc(-n3c4ccccc4c4ccccc43)cc1C21c2ccccc2Oc2c(-n3c4ccccc4c4ccccc43)cccc21 ; c1ccc2c(c1)Oc1cc(-n3c4ccccc4c4ccccc43)ccc1C21c2ccccc2Oc2c(-n3c4ccccc4c4ccccc43)cccc21 ; c1ccc2c(c1)Oc1c(-n3c4ccccc4c4ccccc43)cccc1C21c2ccccc2Oc2c(-n3c4ccccc4c4ccccc43)cccc21]

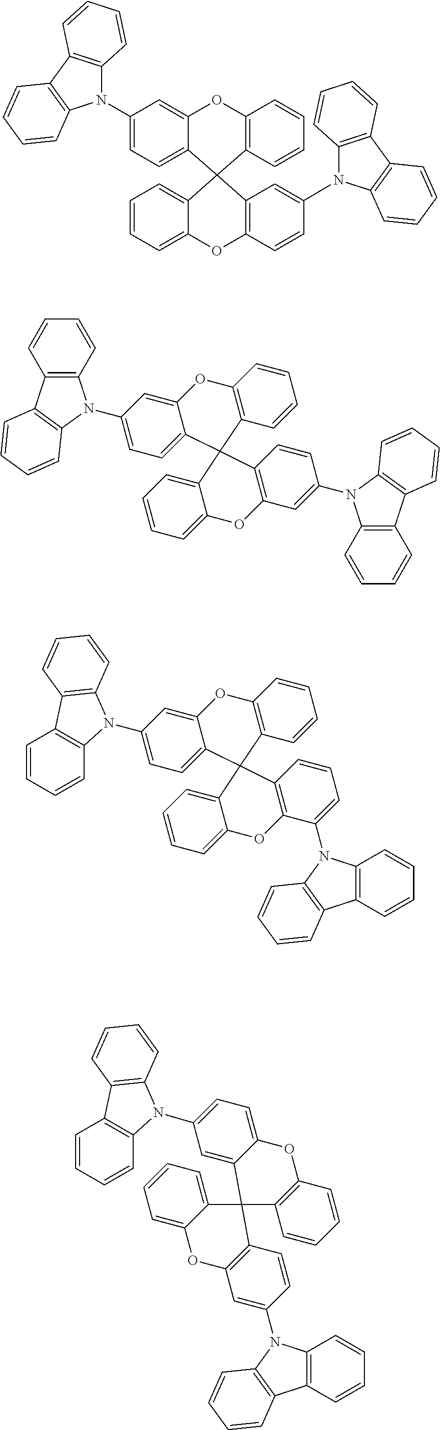 [SMILES: c1ccc2c(c1)Oc1cc(-n3c4ccccc4c4ccccc43)ccc1C21c2ccccc2Oc2ccc(-n3c4ccccc4c4ccccc43)cc21 ; c1ccc2c(c1)Oc1cc(-n3c4ccccc4c4ccccc43)ccc1C21c2ccccc2Oc2cc(-n3c4ccccc4c4ccccc43)ccc21 ; c1ccc2c(c1)Oc1cc(-n3c4ccccc4c4ccccc43)ccc1C21c2ccccc2Oc2c(-n3c4ccccc4c4ccccc43)cccc21 ; c1ccc2c(c1)Oc1cc(-n3c4ccccc4c4ccccc43)ccc1C21c2ccccc2Oc2ccc(-n3c4ccccc4c4ccccc43)cc21]

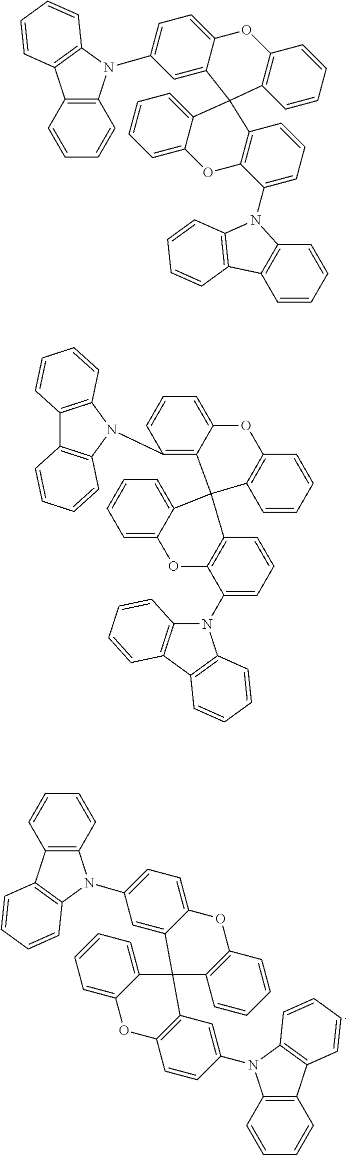 [SMILES: c1ccc2c(c1)Oc1ccc(-n3c4ccccc4c4ccccc43)cc1C21c2ccccc2Oc2c(-n3c4ccccc4c4ccccc43)cccc21 ; c1ccc2c(c1)Oc1cccc(-n3c4ccccc4c4ccccc43)c1C21c2ccccc2Oc2c(-n3c4ccccc4c4ccccc43)cccc21 ; c1ccc2c(c1)Oc1ccc(-n3c4ccccc4c4ccccc43)cc1C21c2ccccc2Oc2ccc(-n3c4ccccc4c4ccccc43)cc21]

It should be noted that this embodiment only takes these four cases as examples, but is not limited thereto.

In addition, an embodiment of the present invention also provides a method of preparing a hole transport material, which includes the following steps:

under a protective gas atmosphere, adding a catalyst to any one or two hydrides of R1, R2, R3, and R4 and halospiroxanthene, which are dissolved in an organic solvent to perform a coupling reaction, to obtain a compound having a chemical structure represented by Formula I:

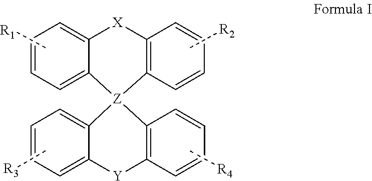

wherein Z includes a carbon atom or a silicon atom, each of X and Y independently includes: an oxygen atom, a sulfur atom,

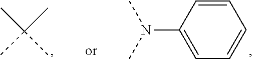

any one or two of R1, R2, R3, and R4 corresponding to the hydrides are electron-donors, and remaining of R1, R2, R3, and R4 other that the electron-donors are hydrogen atoms.

Specifically, a molar ratio of the halospiroxanthene to the hydrides ranges from 1:1 to 1:3, and the method further includes the following steps:

adding a first catalyst, a second catalyst, and a basic substance during the coupling reaction; and

after the coupling reaction is completed, obtaining the compound by cooling, extraction, and column chromatography.

The first catalyst includes a divalent palladium catalyst, the second catalyst includes tri-tert-butylphosphine tetrafluoroborate, the basic substance includes sodium tert-butoxide or potassium tert-butoxide, and the organic solvent includes dehydrated and deoxygenated toluene.

The basic substance provides a basic environment for the reaction to facilitate the reaction.

In addition, each of the any one or two of R1, R2, R3, and R4 corresponding to the hydrides independently includes a carbazolyl or a derivative thereof, diphenylamino or a derivative thereof, phenoxazinyl or a derivative thereof, or acridinyl or a derivative thereof.

Specifically, each of the any one or two of R1, R2, R3, and R4 corresponding to the hydrides independently includes any one of the following groups:

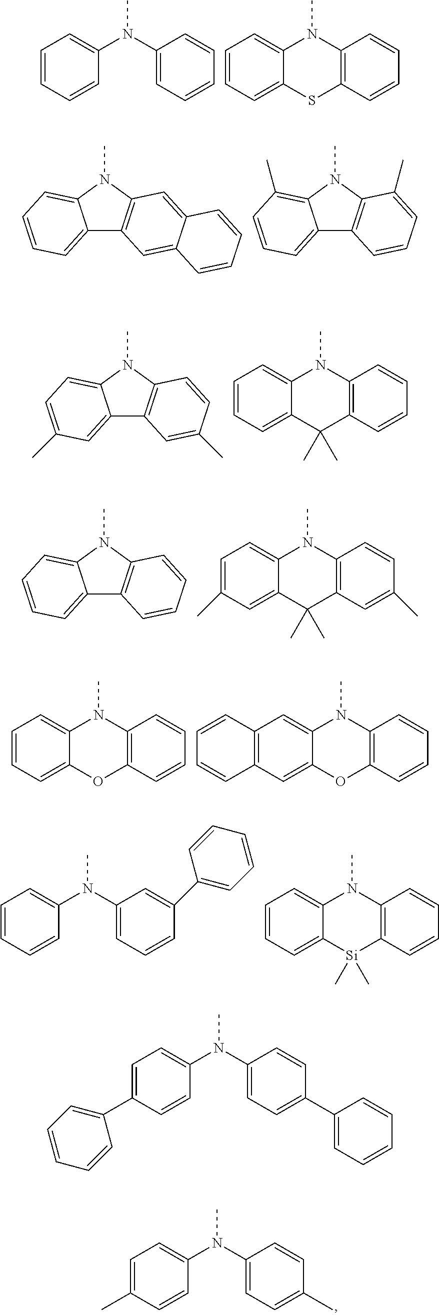

and the hydride includes any one or two of the following compounds:

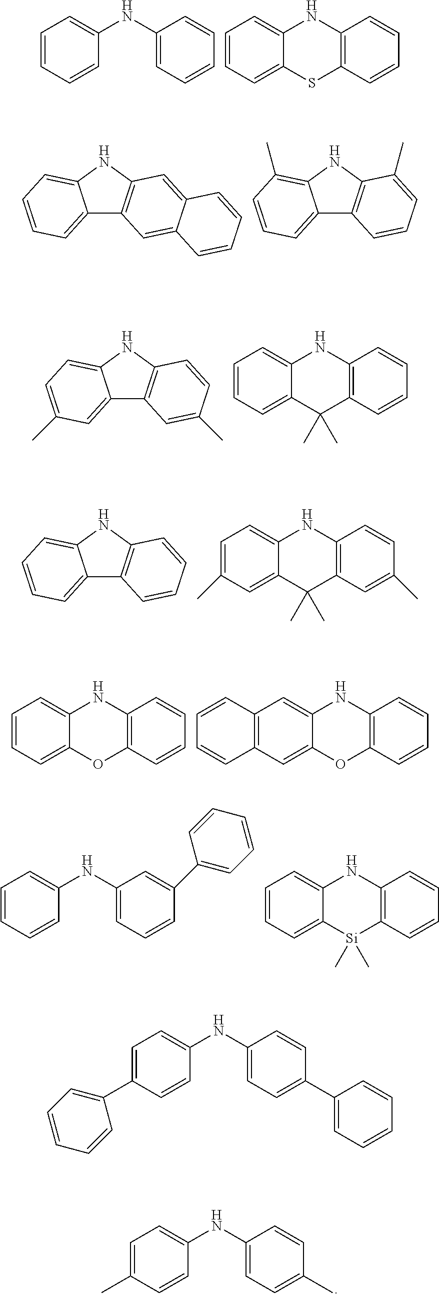

Specifically, the reaction for preparing the compound is described in detail below in combination with the reaction data in the specific examples. It should be noted that the data provided in the examples of the present invention are experimental data, which can be reference data or can be adjusted based on actual needs, and in the following examples, only a part of the structure of the compound is taken as an example for illustration.

### Example 1

The halospiroxaxanthracene is 4,5-dibromo-9,9′-spirobis[oxanthracene], and the hydride is carbazole.

The 4,5-dibromo-9,9′-spirobi[oxanthracene] has a chemical structural formula of

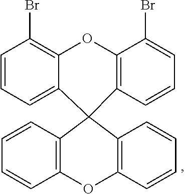 [SMILES: Brc1cccc2c1Oc1c(Br)cccc1C21c2ccccc2Oc2ccccc21]

and a method of preparing 4,5-dibromo-9,9′-spirobi[oxanthracene] is as follows:

Step 1, xanthracene (3.6 g, 20 mmol) and bromosuccinimide (5.28 g, 30 mmol) were added to a 150 ml reactor, and 100 ml of toluene was added thereto.

The reaction was stirred at room temperature for 24 hours. The reaction solution was poured into 300 ml of ice-water, and then extracted with dichloromethane two to three times. The organic extracts were combined and spun dried, and then purified by silica gel column chromatography (dichloromethane:n-hexane, v:v, 1:1), to obtain 1.3 g of 4-bromoxanthene with a yield of 50%.

Mass spectrometry (MS) (EI) m/z: 259.10; 2-bromoxanthene 0.5 g, and yield 19%. MS (EI) m/z: 259.45; 2,4′-dibromoxanthene 0.34 g, yield 11%, and MS (EI) m/z: 337.23.

Scheme of the chemical reaction in the step 1 is as follows:

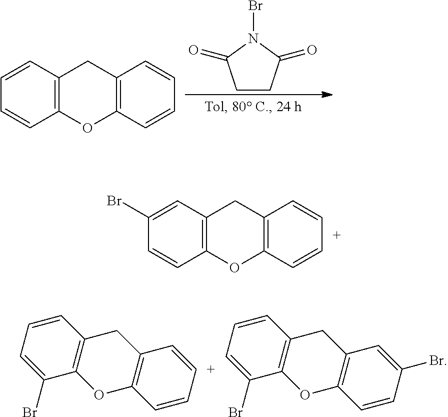

Step 2, 10 mmol of xanthracene and 10 mmol of 4,5-dibromotantrone were added to a 150 ml reactor, and 80 ml of toluene was added thereto.

The reaction was stirred under an argon atmosphere at room temperature for 24 hours. The reaction solution was poured into 300 ml of ice water, and extracted with dichloromethane two or three times. The organic extracts were combined and spun dried, and then purified by silica gel column chromatography (dichloromethane:n-hexane, v:v, 1:5), to obtain 4,5-dibromo-9,9′-spirobi[oxanthracene].

Scheme of the chemical reaction in the step 2 is as follows:

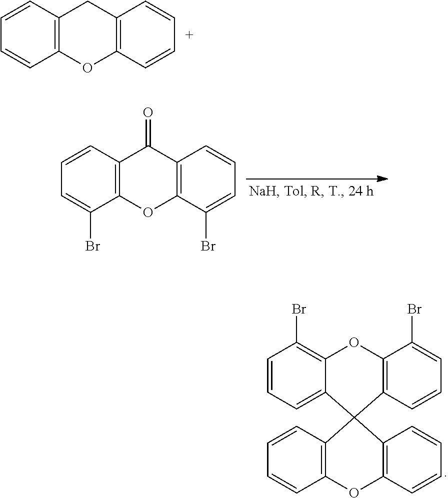

Next, preparation of the compound is conducted, and the preparation process is as follows:

Step 1, 4,5-dibromo-9,9′-spirobi[oxanthracene] (2.52 g, 5 mmol) and carbazole (2.00 g, 12 mmol) were added to a 150 ml reactor, and palladium acetate (90 mg, 0.4 mmol) and tri-tert-butylphosphine tetrafluoroborate (0.34 g, 1.2 mmol) as catalysts were added thereto, and then sodium tert-butoxide (1.16 g, 12 mmol) were added thereto in a glove box, followed by adding 60 ml of previously dehydrated and deoxidized toluene in an argon atmosphere.

A molar ratio of 4,5-dibromo-9,9′-spirobi[oxanthracene] to carbazole is between 1:1 and 1:3, and preferably 1:1.2.

A molar ratio of 4,5-dibromo-9,9′-spirobis[oxanthracene], palladium acetate and tri-tert-butylphosphine tetrafluoroborate is between 1:0.04:0.12 to 1:0.12:0.36, and preferably 1:0.08:0.24.

A molar ratio of 4,5-dibromo-9,9′-spirobi[oxanthracene] to sodium tert-butoxide is between 1:1 and 1:3, and preferably 1:2.4.

A ratio of 4,5-dibromo-9,9′-spirobi[oxanthracene] to the solvent is 1 mmol of 4,5-dibromo-9,9′-spirobi[oxanthracene] corresponding to 8 to 20 ml of toluene solvent, preferably 12 ml.

After that, reaction was performed at 100-160° C. for 12-48 hours, preferably at 120° C. for 24 hours, and cooled to room temperature.

Step 2, the reaction solution was poured into 300 ml of ice-water, and extracted with dichloromethane two or three times. The organic extracts were combined and spun dried, and then purified by silica gel column chromatography (dichloromethane:n-hexane, v:v, 1:1), to obtain 2.8 g of white powder with a yield of 83%. Mass spectrometry analysis MS (EI) m/z: 678.21, the obtained product is one of the structures of the described compounds, which is referred to as Compound 1.

In this Example, the formula of Compound 1 is as follow:

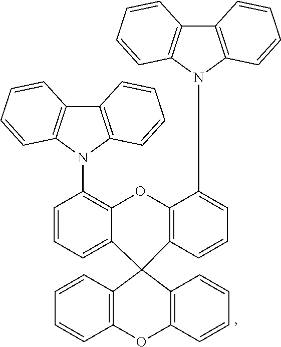 [SMILES: c1ccc2c(c1)Oc1ccccc1C21c2cccc(-n3c4ccccc4c4ccccc43)c2Oc2c(-n3c4ccccc4c4ccccc43)cccc21]

and a scheme of the chemical reaction of preparing Compound 1 is as follow:

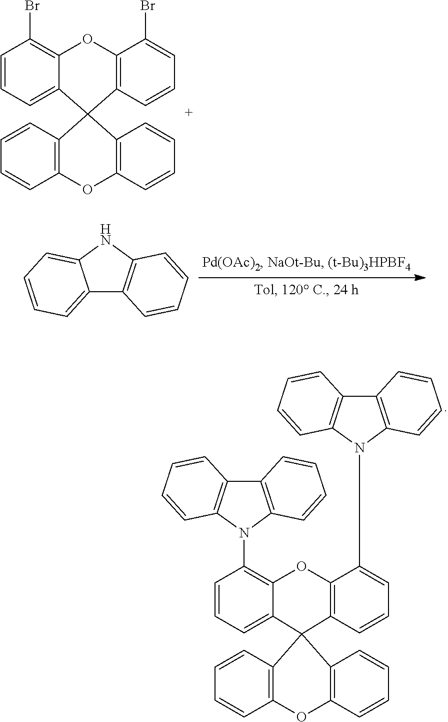 [SMILES: Brc1cccc2c1Oc1c(Br)cccc1C21c2ccccc2Oc2ccccc21 ; c1ccc2c(c1)[nH]c1ccccc12 ; c1ccc2c(c1)Oc1ccccc1C21c2cccc(-n3c4ccccc4c4ccccc43)c2Oc2c(-n3c4ccccc4c4ccccc43)cccc21]

### Example 2

The halospiroxaxanthracene is 2,5-dibromo-9,9′-spirobis[oxanthracene], and the hydride is carbazole.

The 2,5-dibromo-9,9′-spirobi[oxanthracene] has a chemical structural formula of

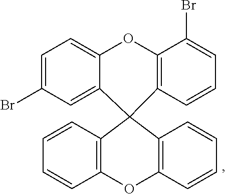 [SMILES: Brc1ccc2c(c1)C1(c3ccccc3Oc3ccccc31)c1cccc(Br)c1O2]

and a method of preparing 2,5-dibromo-9,9′-spirobi[oxanthracene] is as follows:

Step 1, xanthracene (3.6 g, 20 mmol) and bromosuccinimide (5.28 g, 30 mmol) were added to a 150 ml reactor, and 100 ml of toluene was added thereto.

The reaction was stirred at room temperature for 24 hours. The reaction solution was poured into 300 ml of ice-water, and then extracted with dichloromethane two to three times. The organic extracts were combined and spun dried, and then purified by silica gel column chromatography (dichloromethane:n-hexane, v:v, 1:1), to obtain 1.3 g of 4-bromoxanthene with a yield of 50%.

Mass spectrometry (MS) (EI) m/z: 259.10; 2-bromoxanthene 0.5 g, and yield 19%. MS (EI) m/z: 259.45; 2,4′-dibromoxanthene 0.34 g, yield 11%, and MS (EI) m/z: 337.23.

Scheme of the chemical reaction in the step 1 is as follows:

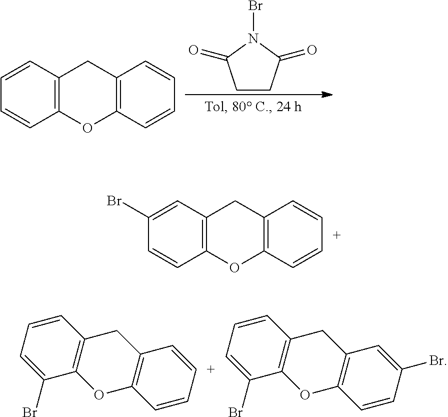

Step 2, 10 mmol of 2,4-dibromoxanthene and 10 mmol of zanthone were added to a 150 ml reactor, and 80 ml of toluene was added thereto.

The reaction was stirred under an argon atmosphere at room temperature for 24 hours. The reaction solution was poured into 300 ml of ice water, and extracted with dichloromethane two or three times. The organic extracts were combined and spun dried, and then purified by silica gel column chromatography (dichloromethane:n-hexane, v:v, 1:5), to obtain 2,5-dibromo-9,9′-spirobi[oxanthracene].

Scheme of the chemical reaction in the step 2 is as follows:

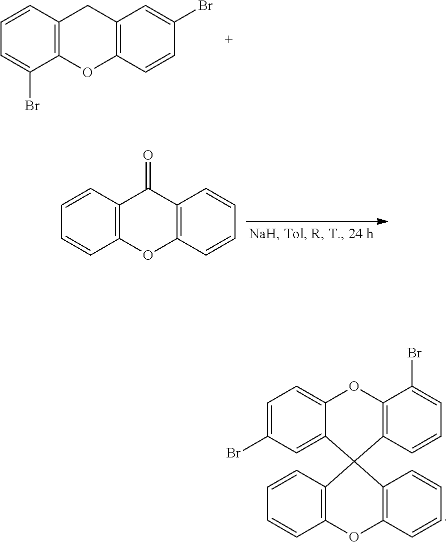 [SMILES: Brc1ccc2c(c1)Cc1cccc(Br)c1O2 ; O=c1c2ccccc2oc2ccccc12 ; Brc1ccc2c(c1)C1(c3ccccc3Oc3ccccc31)c1cccc(Br)c1O2]

Next, preparation of the compound is conducted, and the preparation process is as follows:

Step 1, 2,5-dibromo-9,9′-spirobi[oxanthracene] (2.52 g, 5 mmol) and carbazole (2.00 g, 12 mmol) were added to a 150 ml reactor, and palladium acetate (90 mg, 0.4 mmol) and tri-tert-butylphosphine tetrafluoroborate (0.34 g, 1.2 mmol) as catalysts were added thereto, and then sodium tert-butoxide (1.16 g, 12 mmol) were added thereto in a glove box, followed by adding 60 ml of previously dehydrated and deoxidized toluene in an argon atmosphere.

A molar ratio of 2,5-dibromo-9,9′-spirobi[oxanthracene] to carbazole is between 1:1 and 1:3, and preferably 1:1.2.

A molar ratio of 2,5-dibromo-9,9′-spirobis[oxanthracene], palladium acetate and tri-tert-butylphosphine tetrafluoroborate is between 1:0.04:0.12 to 1:0.12:0.36, and preferably 1:0.08:0.24.

A molar ratio of 2,5-dibromo-9,9′-spirobi[oxanthracene] to sodium tert-butoxide is between 1:1 and 1:3, and preferably 1:2.4.

A ratio of 2,5-dibromo-9,9′-spirobi[oxanthracene] to the solvent is 1 mmol of 2,5-dibromo-9,9′-spirobi[oxanthracene] corresponding to 8 to 20 ml of toluene solvent, preferably 12 ml.

After that, reaction was performed at 100-160° C. for 12-48 hours, preferably at 120° C. for 24 hours, and cooled to room temperature.

Step 2, the reaction solution was poured into 300 ml of ice-water, and extracted with dichloromethane two or three times. The organic extracts were combined and spun dried, and then purified by silica gel column chromatography (dichloromethane:n-hexane, v:v, 1:1), to obtain 2.7 g of white powder with a yield of 80%. Mass spectrometry analysis MS (EI) m/z: 678.11, the obtained product is one of the structures of the described compounds, which is referred to as Compound 2.

In this Example, the structural formula of Compound 2 is as follow:

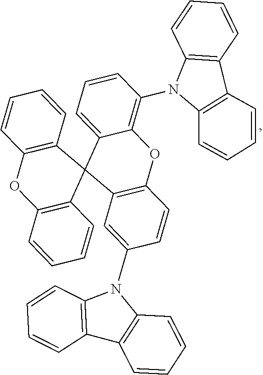 [SMILES: c1ccc2c(c1)Oc1ccccc1C21c2cc(-n3c4ccccc4c4ccccc43)ccc2Oc2c(-n3c4ccccc4c4ccccc43)cccc21]

and a scheme of the chemical reaction of preparing Compound 2 is as follow:

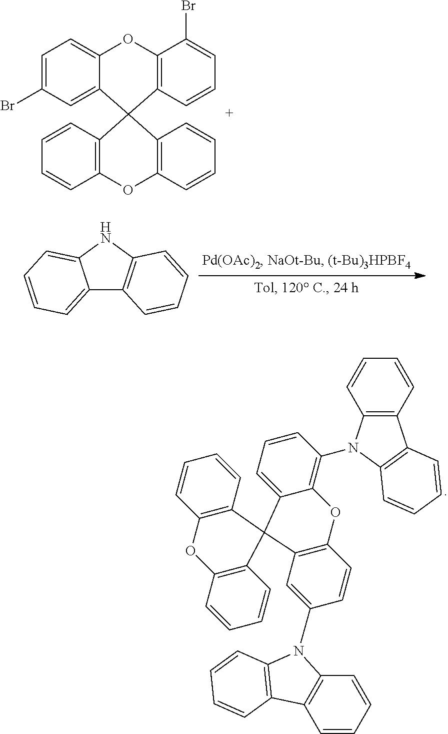 [SMILES: Brc1ccc2c(c1)C1(c3ccccc3Oc3ccccc31)c1cccc(Br)c1O2 ; c1ccc2c(c1)[nH]c1ccccc12 ; c1ccc2c(c1)Oc1ccccc1C21c2cc(-n3c4ccccc4c4ccccc43)ccc2Oc2c(-n3c4ccccc4c4ccccc43)cccc21]

### Example 3

The halospiroxanthracene is 2,7-dibromo-9,9′-spirobis[oxanthracene], and the hydride is carbazole.

The 2,7-dibromo-9,9′-spirobi[oxanthracene] has a chemical structural formula of

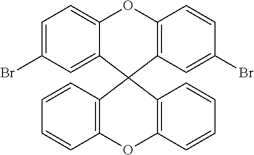 [SMILES: Brc1ccc2c(c1)C1(c3ccccc3Oc3ccccc31)c1cc(Br)ccc1O2]

A method of preparing 2,7-dibromo-9,9′-spirobi[oxanthracene] is as follows:

Step 1, xanthracene (3.6 g, 20 mmol) and bromosuccinimide (5.28 g, 30 mmol) were added to a 150 ml reactor, and 100 ml of toluene was added thereto.

The reaction was stirred at room temperature for 24 hours. The reaction solution was poured into 300 ml of ice-water, and then extracted with dichloromethane two to three times. The organic extracts were combined and spun dried, and then purified by silica gel column chromatography (dichloromethane:n-hexane, v:v, 1:1), to obtain 1.3 g of 4-bromoxanthene with a yield of 50%.

Mass spectrometry (MS) (EI) m/z: 259.10; 2-bromoxanthene 0.5 g, and yield 19%. MS (EI) m/z: 259.45; 2,4′-dibromoxanthene 0.34 g, yield 11%, and MS (EI) m/z: 337.23.

Scheme of the chemical reaction in the step 1 is as follows:

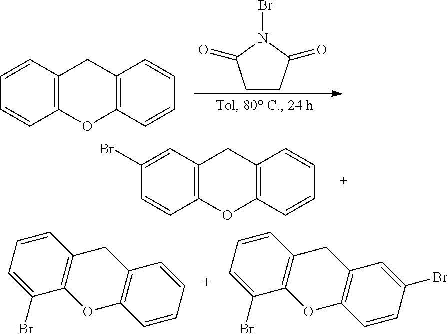

Step 2, 10 mmol of xanthene and 10 mmol of 2,7-dibromozentone were added to a 150 ml reactor, and 80 ml of toluene was added thereto.

The reaction was stirred under an argon atmosphere at room temperature for 24 hours. The reaction solution was poured into 300 ml of ice water, and extracted with dichloromethane two or three times. The organic extracts were combined and spun dried, and then purified by silica gel column chromatography (dichloromethane:n-hexane, v:v, 1:5), to obtain 2,7-dibromo-9,9′-spirobi[oxanthracene].

Scheme of the chemical reaction in the step 2 is as follows:

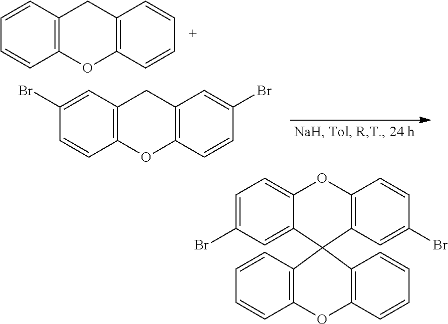

Next, preparation of the compound is conducted, and the preparation process is as follows:

Step 1, 2,7-dibromo-9,9′-spirobi[oxanthracene] (2.52 g, 5 mmol) and carbazole (2.00 g, 12 mmol) were added to a 150 ml reactor, and palladium acetate (90 mg, 0.4 mmol) and tri-tert-butylphosphine tetrafluoroborate (0.34 g, 1.2 mmol) as catalysts were added thereto, and then sodium tert-butoxide (1.16 g, 12 mmol) were added thereto in a glove box, followed by adding 60 ml of previously dehydrated and deoxidized toluene in an argon atmosphere.

A molar ratio of 2,7-dibromo-9,9′-spirobi[oxanthracene] to carbazole is between 1:1 and 1:3, and preferably 1:1.2.

A molar ratio of 2,7-dibromo-9,9′-spirobis[oxanthracene], palladium acetate and tri-tert-butylphosphine tetrafluoroborate is between 1:0.04:0.12 to 1:0.12:0.36, and preferably 1:0.08:0.24.

A molar ratio of 2,7-dibromo-9,9′-spirobi[oxanthracene] to sodium tert-butoxide is between 1:1 and 1:3, and preferably 1:2.4.

A ratio of 2,7-dibromo-9,9′-spirobi[oxanthracene] to the solvent is 1 mmol of 2,7-dibromo-9,9′-spirobi[oxanthracene] corresponding to 8 to 20 ml of toluene solvent, preferably 12 ml.

After that, reaction was performed at 100-160° C. for 12-48 hours, preferably at 120° C. for 24 hours, and cooled to room temperature.

Step 2, the reaction solution was poured into 300 ml of ice-water, and extracted with dichloromethane two or three times. The organic extracts were combined and spun dried, and then purified by silica gel column chromatography (dichloromethane:n-hexane, v:v, 1:1), to obtain 2 g of white powder with a yield of 59%. Mass spectrometry analysis MS (EI) m/z: 678.13, the obtained product is one of the structures of the described compounds, which is referred to as Compound 3.

In this Example, the structural formula of Compound 3 is as follow:

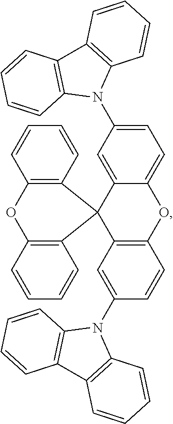 [SMILES: c1ccc2c(c1)Oc1ccccc1C21c2cc(-n3c4ccccc4c4ccccc43)ccc2Oc2ccc(-n3c4ccccc4c4ccccc43)cc21]

and a scheme of the chemical reaction of preparing Compound 3 is as follow:

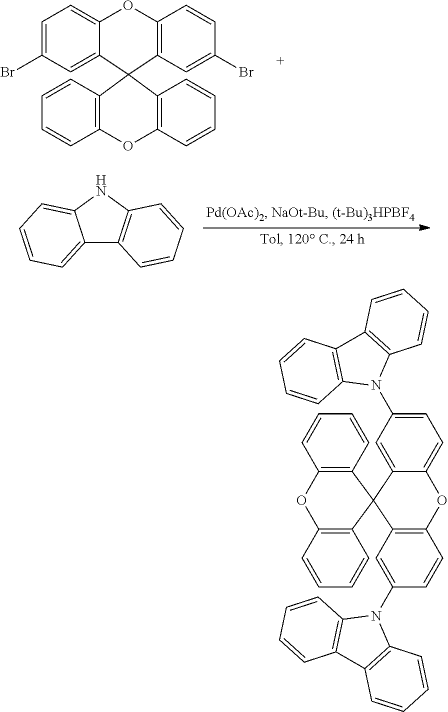 [SMILES: Brc1ccc2c(c1)C1(c3ccccc3Oc3ccccc31)c1cc(Br)ccc1O2 ; c1ccc2c(c1)[nH]c1ccccc12 ; c1ccc2c(c1)Oc1ccccc1C21c2cc(-n3c4ccccc4c4ccccc43)ccc2Oc2ccc(-n3c4ccccc4c4ccccc43)cc21]

### Example 4

The halospiroxaxanthracene is 3,4′-dibromo-9,9′-spirobis[oxanthracene], and the hydride is carbazole.

The 3,4′-dibromo-9,9′-spirobi[oxanthracene] has a chemical structural formula of

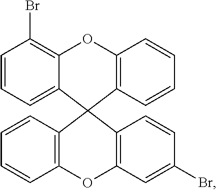 [SMILES: Brc1ccc2c(c1)Oc1ccccc1C21c2ccccc2Oc2c(Br)cccc21]

and a method of preparing 3,4′-dibromo-9,9′-spirobi[oxanthracene] is as follows:

Step 1, xanthracene (3.6 g, 20 mmol) and bromosuccinimide (5.28 g, 30 mmol) were added to a 150 ml reactor, and 100 ml of toluene was added thereto.

The reaction was stirred at room temperature for 24 hours. The reaction solution was poured into 300 ml of ice-water, and then extracted with dichloromethane two to three times. The organic extracts were combined and spun dried, and then purified by silica gel column chromatography (dichloromethane:n-hexane, v:v, 1:1), to obtain 1.3 g of 4-bromoxanthene with a yield of 50%.

Mass spectrometry (MS) (EI) m/z: 259.10; 2-bromoxanthene 0.5 g, and yield 19%. MS (EI) m/z: 259.45; 2,4′-dibromoxanthene 0.34 g, yield 11%, and MS (EI) m/z: 337.23.

Scheme of the chemical reaction in the step 1 is as follows:

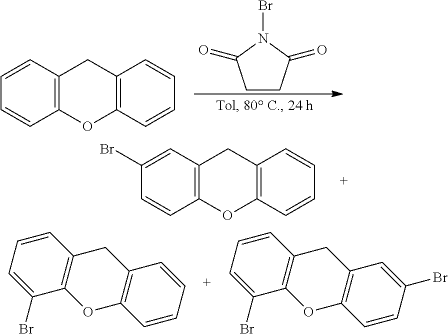

Step 2, 10 mmol of 4-bromoxanthene and 10 mmol of 2-bromozetanone were added to a 150 ml reactor, and 80 ml of toluene was added thereto.

The reaction was stirred under an argon atmosphere at room temperature for 24 hours. The reaction solution was poured into 300 ml of ice water, and extracted with dichloromethane two or three times. The organic extracts were combined and spun dried, and then purified by silica gel column chromatography (dichloromethane:n-hexane, v:v, 1:5), to obtain 3,4′-dibromo-9,9′-spirobi[oxanthracene].

Scheme of the chemical reaction in the step 2 is as follows:

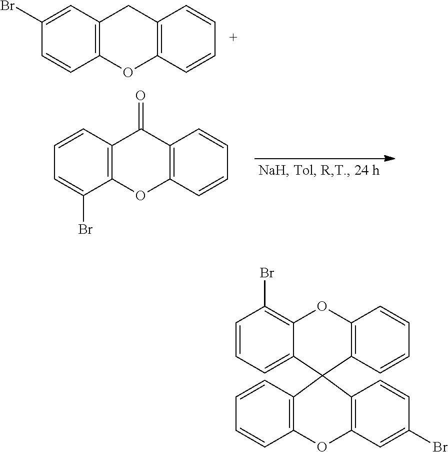

Next, preparation of the compound is conducted, and the preparation process is as follows:

Step 1, 3,4′-dibromo-9,9′-spirobi[oxanthracene] (2.52 g, 5 mmol) and carbazole (2.00 g, 12 mmol) were added to a 150 ml reactor, and palladium acetate (90 mg, 0.4 mmol) and tri-tert-butylphosphine tetrafluoroborate (0.34 g, 1.2 mmol) as catalysts were added thereto, and then sodium tert-butoxide (1.16 g, 12 mmol) were added thereto in a glove box, followed by adding 60 ml of previously dehydrated and deoxidized toluene in an argon atmosphere.

A molar ratio of 3,4′-dibromo-9,9′-spirobi[oxanthracene] to carbazole is between 1:1 and 1:3, and preferably 1:1.2.

A molar ratio of 3,4′-dibromo-9,9′-spirobis[oxanthracene], palladium acetate and tri-tert-butylphosphine tetrafluoroborate is between 1:0.04:0.12 to 1:0.12:0.36, and preferably 1:0.08:0.24.

A molar ratio of 3,4′-dibromo-9,9′-spirobi[oxanthracene] to sodium tert-butoxide is between 1:1 and 1:3, and preferably 1:2.4.

A ratio of 3,4′-dibromo-9,9′-spirobi[oxanthracene] to the solvent is 1 mmol of 3,4′-dibromo-9,9′-spirobi[oxanthracene] corresponding to 8 to 20 ml of toluene solvent, preferably 12 ml.

After that, reaction was performed at 100-160° C. for 12-48 hours, preferably at 120° C. for 24 hours, and cooled to room temperature.

Step 2, the reaction solution was poured into 300 ml of ice-water, and extracted with dichloromethane two or three times. The organic extracts were combined and spun dried, and then purified by silica gel column chromatography (dichloromethane:n-hexane, v:v, 1:1), to obtain 2.4 g of white powder with a yield of 71%. Mass spectrometry analysis MS (EI) m/z: 678.49, the obtained product is one of the structures of the described compounds, which is referred to as Compound 4.

In this Example, the structural formula of Compound 4 is as follow:

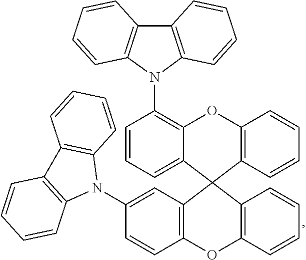 [SMILES: c1ccc2c(c1)Oc1ccc(-n3c4ccccc4c4ccccc43)cc1C21c2ccccc2Oc2c(-n3c4ccccc4c4ccccc43)cccc21]

and a scheme of the chemical reaction of preparing Compound 4 is as follow:

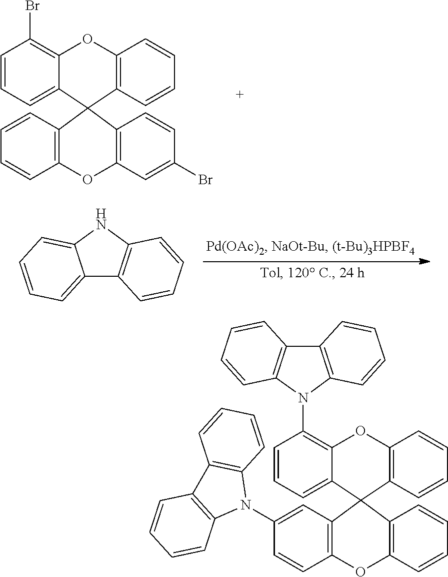 [SMILES: Brc1ccc2c(c1)Oc1ccccc1C21c2ccccc2Oc2c(Br)cccc21 ; c1ccc2c(c1)[nH]c1ccccc12 ; c1ccc2c(c1)Oc1ccc(-n3c4ccccc4c4ccccc43)cc1C21c2ccccc2Oc2c(-n3c4ccccc4c4ccccc43)cccc21]

### Example 5

The 4,4′-dibromo-9,9′-spirobi[oxanthracene] has a chemical structural formula of

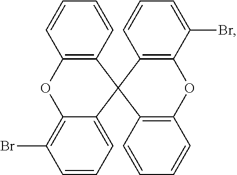 [SMILES: Brc1cccc2c1Oc1ccccc1C21c2ccccc2Oc2c(Br)cccc21]

and a method of preparing 4,4′-dibromo-9,9′-spirobi[oxanthracene] is as follows:

Step 1, xanthracene (3.6 g, 20 mmol) and bromosuccinimide (5.28 g, 30 mmol) were added to a 150 ml reactor, and 100 ml of toluene was added.

The reaction was stirred at room temperature for 24 hours. The reaction solution was poured into 300 ml of ice-water, and then extracted with dichloromethane two to three times. The organic extracts were combined and spun dried, and then purified by silica gel column chromatography (dichloromethane:n-hexane, v:v, 1:1), to obtain 1.3 g of 4-bromoxanthene with a yield of 50%.

Mass spectrometry (MS) (EI) m/z: 259.10; 2-bromoxanthene 0.5 g, and yield 19%. MS (EI) m/z: 259.45; 2,4′-dibromoxanthene 0.34 g, yield 11%, and MS (EI) m/z: 337.23.

Scheme of the chemical reaction in the step 1 is as follows:

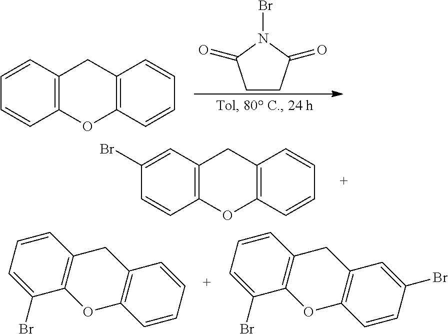

Step 2, 10 mmol of 2-bromoxanthene and 10 mmol of 2-bromozetanone were added to a 150 ml reactor, and 80 ml of toluene was added thereto.

The reaction was stirred under an argon atmosphere at room temperature for 24 hours. The reaction solution was poured into 300 ml of ice water, and extracted with dichloromethane two or three times. The organic extracts were combined and spun dried, and then purified by silica gel column chromatography (dichloromethane:n-hexane, v:v, 1:5), to obtain 2,5-dibromo-9,9′-spirobi[oxanthracene].

Scheme of the chemical reaction in the step 2 is as follows:

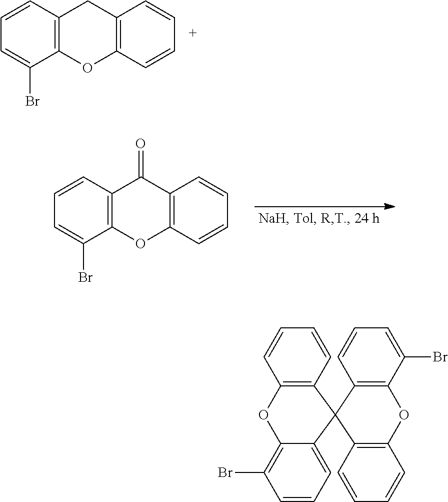 [SMILES: Brc1cccc2c1Oc1ccccc1C2 ; O=c1c2ccccc2oc2c(Br)cccc12 ; Brc1cccc2c1Oc1ccccc1C21c2ccccc2Oc2c(Br)cccc21]

Next, preparation of the compound is conducted, and the preparation process is as follows:

Step 1, 4,4′-dibromo-9,9′-spirobi[oxanthracene] (2.52 g, 5 mmol) and carbazole (2.00 g, 12 mmol) were added to a 150 ml reactor, and palladium acetate (90 mg, 0.4 mmol) and tri-tert-butylphosphine tetrafluoroborate (0.34 g, 1.2 mmol) as catalysts were added thereto, and then sodium tert-butoxide (1.16 g, 12 mmol) were added thereto in a glove box, followed by adding 60 ml of previously dehydrated and deoxidized toluene in an argon atmosphere.

A molar ratio of 4,4′-dibromo-9,9′-spirobi[oxanthracene] to carbazole is between 1:1 and 1:3, and preferably 1:1.2.

A molar ratio of 4,4′-dibromo-9,9′-spirobis[oxanthracene], palladium acetate and tri-tert-butylphosphine tetrafluoroborate is between 1:0.04:0.12 to 1:0.12:0.36, and preferably 1:0.08:0.24.

A molar ratio of 4,4′-dibromo-9,9′-spirobi[oxanthracene] to sodium tert-butoxide is between 1:1 and 1:3, and preferably 1:2.4.

A ratio of 4,4′-dibromo-9,9′-spirobi[oxanthracene] to the solvent is 1 mmol of 4,4′-dibromo-9,9′-spirobi[oxanthracene] corresponding to 8 to 20 ml of toluene solvent, preferably 12 ml.

After that, reaction was performed at 100-160° C. for 12-48 hours, preferably at 120° C. for 24 hours, and cooled to room temperature.

Step 2, the reaction solution was poured into 300 ml of ice-water, and extracted with dichloromethane two or three times. The organic extracts were combined and spun dried, and then purified by silica gel column chromatography (dichloromethane:n-hexane, v:v, 1:1), to obtain 2.6 g of white powder with a yield of 77%. Mass spectrometry analysis MS (EI) m/z: 678.23, the obtained product is one of the structures of the described compounds, which is referred to as Compound 5.

In this Example, the structural formula of Compound 5 is as follow:

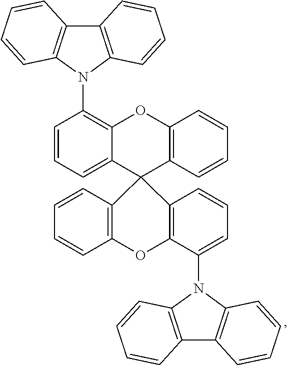 [SMILES: c1ccc2c(c1)Oc1c(-n3c4ccccc4c4ccccc43)cccc1C21c2ccccc2Oc2c(-n3c4ccccc4c4ccccc43)cccc21]

and a scheme of the chemical reaction of preparing Compound 5 is as follow:

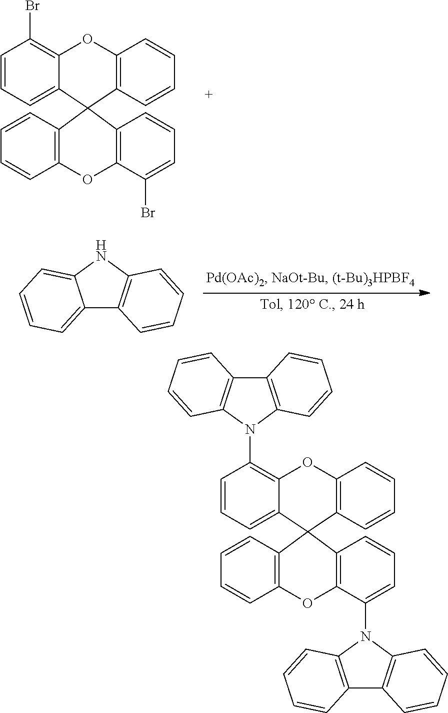

### Example 6

The halospiroxanthracene is 2,2′-dibromo-9,9′-spirobis[oxanthracene], and the hydride is carbazole.

The 2,2′-dibromo-9,9′-spirobi[oxanthracene] has a chemical structural formula of

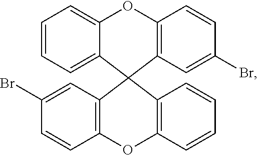 [SMILES: Brc1ccc2c(c1)C1(c3ccccc3O2)c2ccccc2Oc2ccc(Br)cc21]

and a method of preparing 2,2′-dibromo-9,9′-spirobi[oxanthracene] is as follows:

Step 1, xanthracene (3.6 g, 20 mmol) and bromosuccinimide (5.28 g, 30 mmol) were added to a 150 ml reactor, and 100 ml of toluene was added thereto.

The reaction was stirred at room temperature for 24 hours. The reaction solution was poured into 300 ml of ice-water, and then extracted with dichloromethane two to three times. The organic extracts were combined and spun dried, and then purified by silica gel column chromatography (dichloromethane:n-hexane, v:v, 1:1), to obtain 1.3 g of 4-bromoxanthene with a yield of 50%.

Mass spectrometry (MS) (EI) m/z: 259.10; 2-bromoxanthene 0.5 g, and yield 19%. MS (EI) m/z: 259.45; 2,4′-dibromoxanthene 0.34 g, yield 11%, and MS (EI) m/z: 337.23.

Scheme of the chemical reaction in the step 1 is as follows:

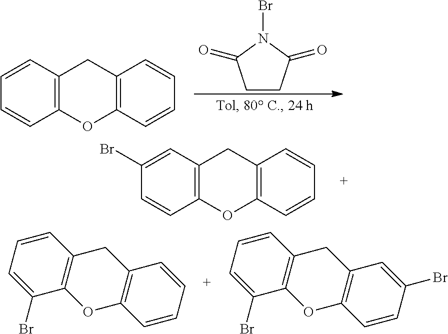

Step 2, 10 mmol of 4-bromoxanthene and 10 mmol of 4-bromozetanone were added to a 150 ml reactor, and 80 ml of toluene was added thereto.

The reaction was stirred under an argon atmosphere at room temperature for 24 hours. The reaction solution was poured into 300 ml of ice water, and extracted with dichloromethane two or three times. The organic extracts were combined and spun dried, and then purified by silica gel column chromatography (dichloromethane:n-hexane, v:v, 1:5), to obtain 2,5-dibromo-9,9′-spirobi[oxanthracene].

Scheme of the chemical reaction in the step 2 is as follows:

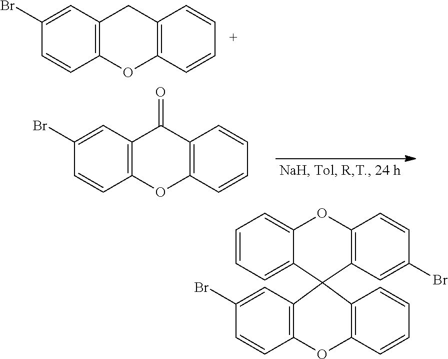

Next, preparation of the compound is conducted, and the preparation process is as follows:

Step 1, 2,2′-dibromo-9,9′-spirobi[oxanthracene] (2.52 g, 5 mmol) and carbazole (2.00 g, 12 mmol) were added to a 150 ml reactor, and palladium acetate (90 mg, 0.4 mmol) and tri-tert-butylphosphine tetrafluoroborate (0.34 g, 1.2 mmol) as catalysts were added thereto, and then sodium tert-butoxide (1.16 g, 12 mmol) were added thereto in a glove box, followed by adding 60 ml of previously dehydrated and deoxidized toluene in an argon atmosphere.

A molar ratio of 2,2′-dibromo-9,9′-spirobi[oxanthracene] to carbazole is between 1:1 and 1:3, and preferably 1:1.2.

A molar ratio of 2,2′-dibromo-9,9′-spirobis[oxanthracene], palladium acetate and tri-tert-butylphosphine tetrafluoroborate is between 1:0.04:0.12 to 1:0.12:0.36, and preferably 1:0.08:0.24.

A molar ratio of 2,2′-dibromo-9,9′-spirobi[oxanthracene] to sodium tert-butoxide is between 1:1 and 1:3, and preferably 1:2.4.

A ratio of 2,2′-dibromo-9,9′-spirobi[oxanthracene] to the solvent is 1 mmol of 2,2′-dibromo-9,9′-spirobi[oxanthracene] corresponding to 8 to 20 ml of toluene solvent, preferably 12 ml.

After that, reaction was performed at 100-160° C. for 12-48 hours, preferably at 120° C. for 24 hours, and cooled to room temperature.

Step 2, the reaction solution was poured into 300 ml of ice-water, and extracted with dichloromethane two or three times. The organic extracts were combined and spun dried, and then purified by silica gel column chromatography (dichloromethane:n-hexane, v:v, 1:1), to obtain 2.2 g of white powder with a yield of 65%. Mass spectrometry analysis MS (EI) m/z: 678.17, the obtained product is one of the structures of the described compounds, which is referred to as Compound 6.

In this Example, the structural formula of Compound 6 is as follow:

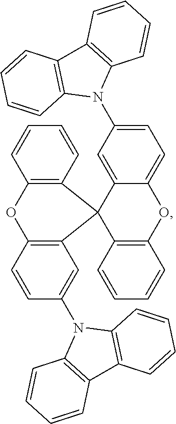 [SMILES: c1ccc2c(c1)Oc1ccc(-n3c4ccccc4c4ccccc43)cc1C21c2ccccc2Oc2ccc(-n3c4ccccc4c4ccccc43)cc21]

and a scheme of the chemical reaction of preparing Compound 6 is as follow:

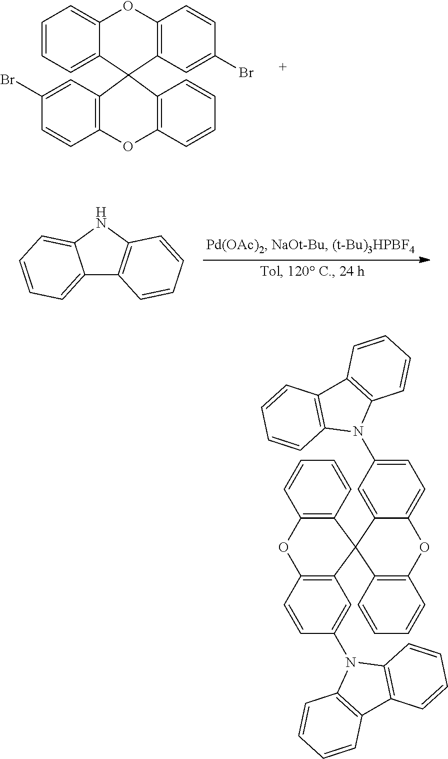 [SMILES: Brc1ccc2c(c1)C1(c3ccccc3O2)c2ccccc2Oc2ccc(Br)cc21 ; c1ccc2c(c1)[nH]c1ccccc12 ; c1ccc2c(c1)Oc1ccc(-n3c4ccccc4c4ccccc43)cc1C21c2ccccc2Oc2ccc(-n3c4ccccc4c4ccccc43)cc21]

In summary, the six structures of the described compounds and their respective preparation processes are provided in the examples of the present invention. It should be noted that the present invention only uses these six structures as examples for illustration, the remaining structures of the compounds are all similar thereto, and the reaction data mentioned therein are for reference only and the present invention is not limited thereto.

The compound provided in an embodiment of the present invention is used as a hole transport material, so that the hole transport material has a higher hole mobility and a suitable energy level, such that the light-emitting efficiency of the organic light-emitting device is improved, and the display effect is elevated.

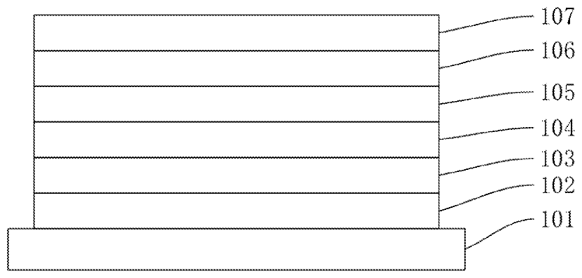

In addition, an embodiment of the present invention further provides an organic light-emitting device. As shown in FIG. 1, the organic light-emitting device includes a first electrode 101, a second electrode 107, and a functional layer disposed between the first electrode 101 and the second electrode 107, and the functional layer includes the hole transport material in the above embodiments.

The functional layer includes a hole injection layer 102, a hole transport layer 103, a light-emitting layer 104, an electron transport layer 105, and an electron injection layer 106.

The first electrode 101 is disposed on a glass substrate, and the first electrode 101 is made of a material including indium tin oxide.

The hole injection layer 102 is disposed on the first electrode 101, and the hole injection layer 102 is made of a material including 1,4,5,8,9,11-hexaazabenzonitrile, and is configured to inject holes into the organic light-emitting device.

The hole transport layer 103 includes the hole transport material, and is disposed on the hole injection layer 102 and configured to transport holes injected by the hole injection layer 102 to the light-emitting layer 104.

The light-emitting layer 104 is disposed on the hole transport layer 103. The light-emitting layer 104 is made of a material including bis[2-((oxo)diphenylphosphino)phenyl]ether and a thermally activated delayed fluorescent material. The light-emitting principle of the light-emitting layer 104 is that holes and electrons recombine in the light-emitting layer 104 to transfer energy to the light-emitting material, so that the light-emitting material is excited to an excited state, and spontaneously returns to a ground state from the excited state, to emit blue light by radiative transitions.

The electron transport layer 105 is disposed on the light-emitting layer 104. The electron transport layer 105 is made of a material including 1,3,5-tris (3-(3-pyridyl) phenyl)benzene, and is configured to inject electrons into the light-emitting layer 104.

The electron injection layer 106 is disposed on the electron transport layer 105. The electron injection layer 106 is made of a material including lithium fluoride, and is configured to inject electrons into the organic light-emitting device.

The second electrode 107 is disposed on the electron injection layer 106, and the second electrode 107 is made of a material including aluminum.

The organic light-emitting device provided by the embodiment of the present invention includes the hole transport material, which has a higher hole mobility and a suitable energy level, and embodiments of the present invention provides a performance data table of the organic light-emitting devices shown in Table 1, including Devices 1 to 7, wherein the hole transport material in Device 1 includes 2,2′-diphenylspiroline, that is, DPA-spiro, and the hole transport materials in Devices 2 to 7 include respectively Compound 1, Compound 2, Compound 3, Compound 4, Compound 5, and Compound 6 provided by the examples of the invention.

The current-brightness-voltage characteristics of the devices were measured by a Keithley source measurement system (Keithley 2400 Sourcemeter, Keithley 2000 Currentmeter) with a calibrated silicon photodiode, and the electroluminescence spectrum was measured by a spectrometer. All measurements were done at room temperature.

Specific performance data are shown in Table 1.

TABLE 1
Performance table of organic light-emitting devices

| | hole | highest | EL | maximum |
|---|---|---|---|---|
| | transport | current | peak | external quantum |
| Device | layer | efficiency (cd/A) | (nm) | efficiency (%) |
| Device 1 | DPA-spiro | 25.6 | 470 | 19.1% |
| Device 2 | Compound 1 | 26.3 | 470 | 20.4% |
| Device 3 | Compound 2 | 29.6 | 470 | 23.1% |
| Device 4 | Compound 3 | 28.1 | 470 | 21.8% |
| Device 5 | Compound 4 | 27.6 | 470 | 21.3% |
| Device 6 | Compound 5 | 28.4 | 470 | 22.6% |
| Device 7 | Compound 6 | 25.8 | 470 | 16.4% |

It can be seen from Table 1 that the maximum current efficiency and the maximum external quantum efficiency of the organic light-emitting devices prepared by the compounds provided in the examples of the present invention are substantially higher than those of the organic light-emitting device prepared from the reference sample of 2,2′-diphenylspiroline. The devices fully illustrate that the hole transport materials provided by the examples of the present invention have excellent light-emitting performance.

In summary, embodiments of the present invention provide a hole transport material with high efficiency, thereby producing an organic light-emitting device with a long service life, and the organic light-emitting device provided by the embodiments of the present invention can be used in various mobile terminals and function displays.

In the above embodiments, the descriptions of each embodiment have their own emphasis. The parts that are not described in detail in an embodiment can be referred to the detailed descriptions in other embodiments above, which will not be repeated herein for brevity.

The hole transport material, the preparation method thereof, and the organic light emitting device provided in the embodiments of the present invention have been described in detail above. The specific examples are used herein to explain the principles and embodiments of the present invention. The description of the above embodiments is only used to help understand the technical solution of the present invention and its core ideas, and those of ordinary skill in the art should understand that it can still modify the technical solutions described in the foregoing embodiments, or equivalently replace some of the technical features thereof. These modifications or replacements do not make the essence of the corresponding technical solutions depart from the scope of the technical solution of each embodiment of the present invention.

What is claimed is:

1. A hole transport material, comprising a compound having a chemical structure represented by Formula I: 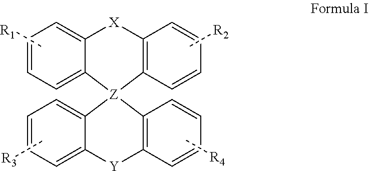
wherein Z comprises a carbon atom or a silicon atom, each of X and Y independently comprises: an oxygen atom, a sulfur atom, 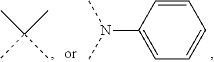
any one or two of R1, R2, R3, and R4 are electron-donors, and remaining of R1, R2, R3, and R4 other that the electron-donors are hydrogen atoms.

2. The hole transport material according to claim 1, wherein each of R1, R2, R3, and R4 independently is or substitutes any hydrogen atom on its corresponding benzene ring.

3. The hole transport material according to claim 1, wherein each of the any one or two of R1, R2, R3, and R4 independently comprises a carbazolyl or a derivative thereof, diphenylamino or a derivative thereof, phenoxazinyl or a derivative thereof, or acridinyl or a derivative thereof.

4. The hole transport material according to claim 3, wherein each of the any one or two of R1, R2, R3, and R4 independently comprises any one of the following groups:

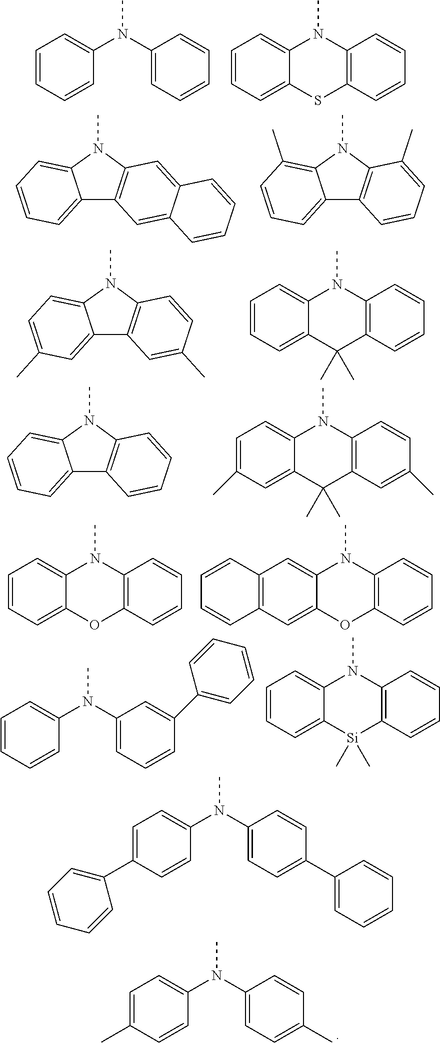

5. A method of preparing a hole transport material, the method comprising the following steps:
under a protective gas atmosphere, adding a catalyst to any one or two hydrides of R1, R2, R3, and R4 and halospiroxanthene, which are dissolved in an organic solvent to perform a coupling reaction, to obtain a compound having a chemical structure represented by Formula I: 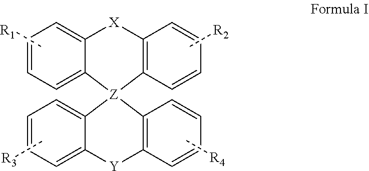
wherein Z comprises a carbon atom or a silicon atom, each of X and Y independently comprises: an oxygen atom, a sulfur atom, 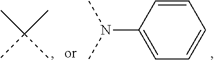
any one or two of R1, R2, R3, and R4 corresponding to the hydrides are electron-donors, and remaining of R1, R2, R3, and R4 other that the electron-donors are hydrogen atoms.

6. The method of preparing the hole transport material according to claim 5, wherein each of the any one or two of R1, R2, R3, and R4 corresponding to the hydrides independently comprises a carbazolyl or a derivative thereof, diphenylamino or a derivative thereof, phenoxazinyl or a derivative thereof, or acridinyl or a derivative thereof.

7. The method of preparing the hole transport material according to claim 6, wherein each of the any one or two of R1, R2, R3, and R4 corresponding to the hydrides independently comprises any one of the following groups:

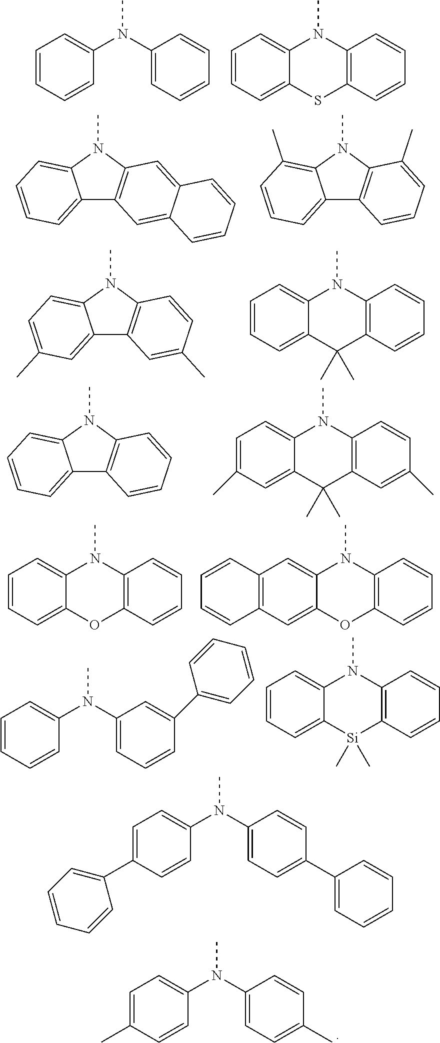

8. The method of preparing the hole transport material according to claim 5, wherein a molar ratio of the halospiroxanthene to the hydrides ranges from 1:1 to 1:3, and the method further comprises the following steps:
adding a first catalyst, a second catalyst, and a basic substance during the coupling reaction; and
after the coupling reaction is completed, obtaining the compound by cooling, extraction, and column chromatography.

9. The method of preparing the hole transport material according to claim 8, wherein the first catalyst comprises a divalent palladium catalyst, the second catalyst comprises tri-tert-butylphosphine tetrafluoroborate, the basic substance comprises sodium tert-butoxide or potassium tert-butoxide, and the organic solvent comprises dehydrated and deoxygenated toluene.

10. An organic light-emitting device, comprising a first electrode, a second electrode, and a functional layer disposed between the first electrode and the second electrode, and the functional layer comprises a hole transport material,
wherein the functional layer comprises:
a hole injection layer disposed on the first electrode;
a hole transport layer comprising the hole transport material and disposed on the hole injection layer;
a light-emitting layer disposed on the hole transport layer;
an electron transport layer disposed on the light-emitting layer; and
an electron injection layer disposed on the electron transport layer;
wherein the hole transport material comprises a compound having a chemical structure represented by Formula I: 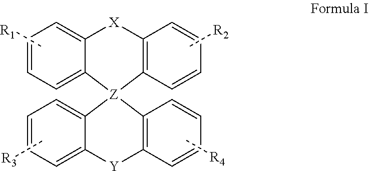
wherein Z comprises a carbon atom or a silicon atom, each of X and Y independently comprises: an oxygen atom, a sulfur atom, 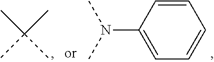
any one or two of R1, R2, R3, and R4 are electron-donors,
and remaining of R1, R2, R3, and R4 other that the electron-donors are hydrogen atoms.

11. The organic light-emitting device according to claim 10, wherein each of R1, R2, R3, and R4 independently is or substitutes any hydrogen atom on its corresponding benzene ring.

12. The organic light-emitting device according to claim 10, wherein each of the any one or two of R1, R2, R3, and R4 independently comprises a carbazolyl or a derivative thereof, diphenylamino or a derivative thereof, phenoxazinyl or a derivative thereof, or acridinyl or a derivative thereof.

13. The organic light-emitting device according to claim 12, wherein each of the any one or two of R1, R2, R3, and R4 independently comprises any one of the following groups: [chemical structure]
